(19) United States
(12) Patent Application Publication
(10) Pub. No.: US 2004/0260163 A1
(43) Pub. Date: Dec. 23, 2004

(54) Non-invasive diagnostic systems for lower urinary tract disorders

(76) Inventors: Reuben E. Kron, Bryn Mawr, PA (US); Mitchell Litt, Philadelphia, PA (US)

Correspondence Address: JONES DAY, 222 EAST 41ST ST, NEW YORK, NY, 10017, US

(21) Appl. No.: 10/489,990

(22) PCT Filed: Sep. 17, 2002
(86) PCT No.: PCT/US02/29342

Related U.S. Application Data
(60) Provisional application No. 60/322,588, filed on Sep. 17, 2001.

(51) Int. Cl. A61B 5/05

(52) U.S. Cl. 600/345

(57) ABSTRACT

An analysis, measurement, and data reduction system for carrying out a noninvasive urodynamic analysis of the human male or female urogenital tract. The apparatus comprises a urethral extender device, a vacuum system, a flow and pressure measuring and control system, a parameter variation device, and a control, acquisition and analysis system capable of the analysis of time dependent urine pressure and flow data so as to obtain clinically significant parameters, including bladder pressure and resistance and compliance parameters for the components of the urogenital tract.

### FIELD OF THE INVENTION

This invention relates to urodynamic devices which non-invasively attach and seal to the urethra of a patient to allow accurate and convenient measurement of both urine flow rate and bladder pressure. The device measures urine released during urination and can be used to determine pathological changes in the structure and function of lower urinary tract components. After noninvasive measurements of urodynamic variables are made, analysis of the data is performed to provide a differential diagnosis to determine which component or components of the lower urinary tract may be responsible for producing specific patient symptoms, disease or to determine whether the patient is at risk of disease or organ dysfunction.

### BACKGROUND OF THE INVENTION

The lower urinary tract (“LUT”) comprises the bladder and the urethra. To date, the diagnosis of disorders of the LUT has required invasive procedures, i.e., the insertion of catheters or other devices into body orifices to make measurements of urine flow rate and pressure during micturition. Except for uroflowmetry (a non-invasive procedure requiring the patient to void against a rotation disk flow transducer to measure flow), most currently available diagnostic procedures applicable to LUT problems are invasive, cumbersome, uncomfortable, expensive and limited to providing urine flow rates without information as to the instantaneous pressure of the urine flow. They also can result in trauma, infection, and perforation of the urethra or bladder, while providing only marginally helpful diagnostic information. For instance, urodynamic procedures requiring the placement of catheters in the bladder give a picture of bladder function, but no information about the degree of urethral obstruction, while urethral flow resistance tests employing multilumen catheters in the urethra are difficult to perform and give ambiguous results. As a result, most urologists depend mainly on clinical findings (enlarged prostate), simple non-invasive uroflow testing, and subjective symptoms such as the patient's score on the American Urological Association symptom checklist for diagnosis. The validity of this self-administered index has been questioned.

In recent years, there have been several attempts to develop practical non-invasive methods to differentially diagnose the causes of LUT disorders. Disorders of the LUT may include benign prostatic hypertrophy; urinary incontinence due to an overactive bladder, neuropathic bladder, prostatectomy, or overflow incontinence; bladder outlet obstruction; urethral stricture; bladder neck dyssynergia; poor detrusor contractility; detrusor-sphincter dyssynergia; and neuropathic bladder dysfunction. Because the urethra in males serves the dual function of urine voiding and semen delivery, its anatomy and physiology are more complex than in the female, and so diagnosis of the male LUT has received much greater attention. In the male, the major problem is to identify the source(s) of decreased urine flow during micturition, and to distinguish the etiology of subjective symptoms such as weak stream, urgency, frequency, etc. These complaints are usually attributed to benign prostatic hypertrophy (“BPH”), with resultant obstruction of the prostatic urethra, a condition usually treated surgically. However, these same symptoms may also be caused by other obstructive conditions or by non-obstructive conditions such as bladder weakness due to neurological disease. In the female, similar symptoms may arise from completely different etiology.

In males, most surgical treatment is currently performed on the basis of clinical symtomology plus non-specific urine flow testing (uroflowmetry). For maximum flow rates below 15 mL/sec the patient may be considered a candidate for an operation to increase the caliber of the prostatic urethra, most often a transurethral prostatectomy (“TURP”). Literature indicates that roughly about $3 billion per year is spent on prostatic surgery, mostly for treatment of presumed BPH. However, only about half of these procedures are successful in improving urine flow and relieving the subjective symptoms of BPH, with the remainder experiencing little or no relief. In addition, a non-trivial percentage of patients have surgical complications including urinary incontinence, sexual impotence, infection, etc. A small mortality rate also accompanies this morbidity. Such a large percentage of unsuccessful procedures with serious post-surgical sequelae indicate a need for more precise and objective diagnostic procedures.

As performed today, uroflowmetry is usually noninvasive. The mass or volume of external urine flow from the urethra is collected as a function of time, and the urine flow rate is calculated by differentiating this function. A number of different clinical devices for doing this test are available and in general use. A method for using only this measure with a sophisticated computer analysis of the flow curve has been patented (U.S. Pat. No. 5,377,101 to Rollema). Another non-invasive method describes a urine drainage and collection device for uroflowmetry (U.S. Pat. No. 5,616,138 to Propp). However, these methods cannot characterize the LUT dynamics because pressure is not measured. Simple kinematic measurements such as flow rate cannot adequately describe and quantitatively model the overall dynamics of a complex, nonlinear flow system having both resistive elements (sphincter, prostate and distal urethra) and compliant elements (bladder and urethra), in which the dimensions are nonlinear functions of both pressure and flow rate. Attempts to create quantitative models had been made for many years, but were largely abandoned some time ago because no adequate noninvasive measurement methods were available to adequately determine the parameters needed.

Recently, several approaches have been tested to provide a differential diagnosis between bladder and urethral sources of lower urinary tract disorders. In one such method, the penis is compressed briefly after voiding begins and then released. Sullivan, Penile urethral compression—release maneuver as a non-invasive screening test for diagnosing prostatic obstruction, 19(6) NEUROUROLOGY AND URODYNAMICS, 657 (2000). The resulting ratio of surge flow rate to steady flow rate is compared with prostatic or bladder outlet obstruction as determined by other means. While significant differences were alleged between test subjects and non-obstructed control subjects, the cohort was small and the statistics were not compelling.

However, two newer methods in which both flow rate and pressure measurements are made have been published. The first method involves the application of a pneumatic occluder, such as an infant blood pressure cuff, around the penis to cut off urine flow during micturition. This method is disclosed in U.S. Pat. Nos. 5,807,278 and 5,823,972, both to McRae. The reduced cuff pressure at which urine flow just resumes is a measure of the static pressure in the bladder. Ranges for normal bladder pressure may be determined and compared with the patient's reading to diagnose a “weak” bladder as a possible source of reduced urine flow. This method may also be adapted to estimate urethral resistance by measuring urine flow rate as a function of pressure with changing degree of occlusion. A more recent study using somewhat different apparatus confirmed that the cuff method gives good agreement with invasive measurements of urethral and bladder pressures. However, the cuff method presents minor problems such as pain at the site of occlusion, and differences in results due to placement, size, and design of the cuff.

The second method, is similar in concept and use to the cuff, but occludes the flow by use of an external catheter, i.e., an incontinence condom with a pressure sensor attached to the exit tube. Occluding the tube distal to the sensor to stop the flow gives a static pressure reading assumed equal to the bladder pressure. See Pel, Non-invasive measurement of bladder pressure using an external catheter, 18(5) NEUROUROLOGY AND URODYNAMICS 455 (1999); Gommer, Validity of a non-invasive determination of the isovolumetric bladder pressure during voiding in men with LUTS, 18(5) NEUROUROLOGY AND URODYNAMICS 477 (1999); Rikken, Repeat noninvasive bladder pressure measurements with an external catheter, 162(2) J. UROLOGY 474 (1999). These studies generally confirm that the occlusion pressure can give a measure of bladder pressure. A more recent study used a series of different resistance tubes, each with a control valve, in the outflow line instead of totally occluding the flow.

Both the cuff and bladder methods represent advances. However, from a quantitative urodynamic point of view, there are a number of deficiencies that limit both the accuracy of measurement and its interpretation. First, meaningful estimates of LUT resistances and compliances require making simultaneous measures of instantaneous pressure and urine flow rate as a function of time. With the systems used, urine flow is measured by collecting the flow volume as a time function and mathematically or numerically calculating the flow rate. Such methods at best will produce a time phase shift between pressure and flow measurements, depending on where and how the pressure measurement is made. Also, since any such urine collection system is noisy, filtering is required, which further biases the flow signal. Such differences can be significant in a rapidly accelerating or decelerating flow during occlusion or resumption of flow. Second, both the cuff and condom methods include compliances of unknown value: the cuff contains a variable amount of air, and the condom, by its material nature, is compliant, in addition to being subject to leaks at high occlusion pressures. To obtain clinically meaningful bladder pressure values, as well as estimates of system resistances and compliances from flow-pressure curves after flow resumes, requires a perfectly non-compliant leak-free seal to the urethra, as well as the ability to measure instantaneous pressure and flow rates at the end of the urethra. Third, since the LUT, especially the distal urethra, is itself highly compliant, backpressure placed in the flow line will change the dimensions of the LUT and lead to false conclusions. Also, none of the systems described to date have any potential for use in females. This invention addresses these and other needs.

### SUMMARY OF THE INVENTION

The urodynamic diagnosis device of the present invention is a novel noninvasive apparatus that can be used in methods for carrying out a complete non-invasive and accurate urodynamic measurement and analysis of the male or female LUT. The invention eliminates the deficiencies of conventional methods, is non-invasive, can be used in males or females, and can provide quantitative, clinically useful measures of the entire LUT function i.e., bladder function, urethra function, prostate function and urine flow. It takes into account nonlinearity of the flow system, due both to non-Poiseullian nature of the flow, as well as the dependence of flow resistances and compliances on changes in geometry with pressure. It provides a non-compliant, leak free seal to the urethral opening in males or females using a novel vacuum attachment technique and/or device. The invention is safe and non-traumatic for the patient clinically, mechanically and electrically. Also, by employing advanced techniques of computer control and analysis, it can rapidly provide an output consisting of objective urodynamic parameters, (including, among others, resistances and compliances for both bladder and urethra) which can be used for a differential diagnosis of the LUT to identify the source and magnitude of abnormal flow conditions. This information can also be used to diagnose prostate function and/or be used to pinpoint the cause of irregular urination including irregular frequency, flow and pain associated with urination.

The urodynamic diagnosis device is comprised of a urethral extender device, a vacuum system, a flow and pressure measuring and control system, a parameter variation device, and a control, acquisition and analysis system. The urethral extender device utilizes vacuum pressure to affix and form a leak free seal to the tissue immediately surrounding the urethra of a patient. The urethral extender device is equally applicable to use on both male and female patients. The male version of the urethra extender device is constructed to conform with the glans penis of a male, and the female version is constructed to conform with tissue surrounding the urethra of a female. A urethral extension tube within the urethral extender device abuts directly against the urethral opening and surrounding tissue on e.g., the head of the penis. A leak free seal/connection is then made from the urethra to the device and subsequent tubing. This allows normal urinary function to occur without altering the pressure or flow rate of urine as it exits the urethra. Most significantly, by using the urethral extender device, testing can proceed in a completely non-invasive manner, thereby eliminating the trauma associated with the insertion of a catheter into the urethra of the patient. The vacuum system provides the required vacuum pressure to maintain the urethral extender device in position and to provide the leak free seal to the urethra from the UED which is connected to measuring devices and/or receptacles.

The urethral extender device also has the capability of being used with existing methods and apparatus to render them non-invasive. In those procedures where a catheter would normally be required, a urethral extender device would be used in place of the existing catheter to provide a less traumatic alternative. In other words, the catheter which is normally connected to a measuring device and/or receptacle is now replaced by a non-invasive UED.

The flow and pressure measuring and control system receives the unaltered urine flow from the urethral extender device to measure urine pressure and flow as a continuous function of time. The flow and pressure measuring and control system also can modify the incoming urine flow rate and pressure to allow for measurements of lower urinary tract function under varying conditions, thereby acquiring differential data to help isolate the source of dysfunction. In other words, resistance can be added to assist in measuring flow rate, flow pressure and bladder or prostate function. A parameter variation device may optionally be included in the system circuit to allow the operator the option of physically adding known external flow elements to provide further differential conditions. This may be useful in certain situations where it may be difficult to obtain precise quantitative differences in parameter values from the natural flow curves.

The control, acquisition and analysis system comprises a computer, software and a power unit to dynamically control the flow and pressure measuring and control system, and thereby alter flow variables with time, according to a preset program. Thus, pressure and flow rate dependent functions can be manipulated by the analysis software to give clinically meaningful urodynamic parameters such as: bladder pressure and measures of flow resistance and compliance in the bladder, prostatic and distal urethra. The computer runs a data analysis and parameter calculation program at the completion of the test which solves flow model equations to provide readout of clinically useful parameters. The output from the system thus can provide the physician with diagnostic information as to the source of lower urinary tract dysfunction by indicating the flow resistance and compliance of the system.

The diagnostic information that has been provided allows the physician to prescribe an appropriate treatment for the symptoms exhibited by the patient. Dysfunction of the prostate can be differentiated from dysfunction of the bladder to prevent unnecessary or improper treatment. Thus, for example, where a conventional uroflowmetry test might indicate BPH (and require a painful transurethral prostatectomy), the present invention would be able to indicate that the reduced flow rate was not due to a problem with the prostate, but rather with the bladder. A more appropriate and effective treatment could then be applied by the physician. Also, an indication of the effectiveness of prior treatments could be assessed by conducting additional tests to determine the trend of LUT functions. In addition, the device also allows the physician to diagnose LUT dysfunctions before they exhibit symptoms. For instance, a bladder outlet obstruction may not be accompanied by a reduced urine flow rate because of the compensation by the bladder to the increased flow resistance. However, the urodynamic diagnosis device would be able to provide an early warning as to the presence of bladder dysfunction, even before noticeable symptoms arose.

In addition, because of the automated nature of the test, the device could be used not only by a physician, but also by trained technicians and nursing staff. The reasonable size and self contained nature of the device also allows utilization outside of the hospital setting, such as in a physician's office or a urology clinic.

Since it is non-invasive, it can be used routinely in the doctor's office on patients at risk of BPH, or other disorders, such as men over 59, men with prostate problems, or men over 40 with a family history of prostate problems gall stones, urinary incontinence, or other LUT disorders.

### BRIEF DESCRIPTION OF THE DRAWINGS

FIG. 1 is a schematic diagram of the elements of the urodynamic diagnosis device.

FIG. 2 is a cutaway diagram of a male urethral extender device.

FIG. 3 is a cutaway diagram of a female urethral extender device.

FIG. 4 is a schematic diagram of the vacuum system of the urodynamic diagnosis device.

FIG. 5 is a schematic diagram of the flow and pressure measuring and control system.

FIG. 6 is a schematic diagram of an alternative flow and pressure measuring and control system.

FIG. 7 is a schematic diagram of a parameter variation device.

FIG. 8 is a schematic diagram of a control, acquisition and analysis subsystem.

FIG. 9 is an example of a pressure-time graph produced by the urodynamic diagnosis device.

FIG. 10 is an example of a pressure and flow rate graph produced by the urodynamic diagnosis device.

FIG. 11 is an example of pressure, volume and flow curves produced by the urodynamic diagnosis device.

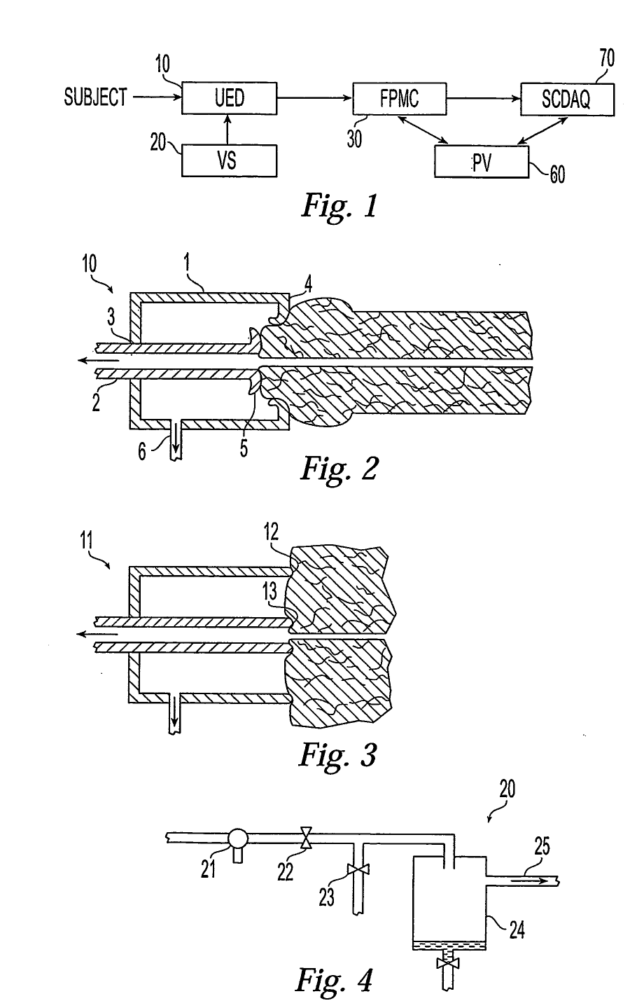

### DETAILED DESCRIPTION OF THE INVENTION

The invention consists of several subsystems acting alone or in concert with each other or other conventional devices; in particular, in concert with the various embodiments of the invention. Generally, the subsystems are connected by liquid and/or air filled tubes and electrical wires, and include various sensors, control valves and electronic/electrical apparatus. All electrical apparatus is shielded from electrical contact with the subject. More than one embodiment of each of the subsystems is described and each may be preferred for different specific subjects or applications.

In order to create a non-invasive, leak free seal to the urethra of a patient, the urodynamic diagnosis device is equipped with a urethral extender device that allows the physician or technician to create a leak free seal and/or vacuum connection to the tissue surrounding the urethra of the patient, without interfering with the urethra itself. The urethral extender device is placed in abutting contact with the tissue surrounding the urethra and a vacuum system is activated to create a leak free seal between the urethral urine flow and the device. An unobstructed flow path is thereby created from the urethra, allowing accurate measurements of urine flow and pressure at the urethral opening. The urethral extender device is effective on both males and females, unlike current analysis devices, and at no time enters into the urethra of the patient.

Once the urethral extender device is attached to the urethra of the patient, the patient begins to urinate. The unobstructed urine flows into a flow and pressure measuring and control system that comprises instrumentation that measures both the instantaneous pressure and flow rate of the urine. Variations to the flow and pressure measuring and control system allow different tests to be performed, all during a single episode of urination. A control, acquisition, and analysis system receives the measurements and communicates control information to the flow and pressure measuring and control system on the basis of the information received. At the conclusion of the test, the control, acquisition, and analysis system provides to the physician diagnostic information about the patient, including the likely cause of LUT dysfunction, to allow the physician to prescribe an appropriate treatment that addresses the actual cause of the dysfunction.

The male Urethral Extender Device (“UED”) 10 is shown in FIG. 2. The preferred embodiment comprises an open-ended vacuum chamber 1 and a urethral extension tube 2, joined at the closed end of vacuum chamber 1 by a pressure tight sliding seal 3. The urethral extension tube 2 extends through the vacuum chamber, and out from the closed end of vacuum chamber 1 a sufficient distance to allow for connection to tubing extending from the flow and pressure measurement device 30, described below.

An outer adaptor ring 4 and an inner adaptor ring 5 are located at, and extend from, the open end of vacuum chamber 1 and urethral extension tube 2, respectively. The outer adaptor ring 4 and inner adaptor ring 5 are positioned in approximate concentric relation to each other. Sliding seal 3 allows the urethral extension tube 2 and the inner adaptor ring 5 to be adjusted in position relative to the vacuum chamber 1 and outer adaptor ring 4, thereby allowing for variation in the size of the opening into which is inserted the tip (glans) of the penis. Sliding seal 3 comprises a threaded nut that surrounds a segment of urethral extension tube 2, and engages a threaded opening at the closed end of vacuum chamber 1 to maintain the relative position of the inner adaptor ring 5 and the outer adaptor ring 4. Surfaces of the adaptor rings 4 and 5 are smooth and shaped to conform to the surface of the glans penis of a patient. The urethral extension tube 2 serves as an extension of the urethra, and allows urine flow from the urethra to pass through without altering either the rate of flow or the pressure of the stream as the urine exits the urethra. Thus, the size and shape of the inner adaptor ring 5 has been designed to permit the urethral opening slit to be fully encompassed, and yet be free to expand under urine flow conditions, thereby adding no resistance to flow.

The vacuum chamber 1 is equipped with a vacuum outlet port 6 that allows a vacuum system 20 to be connected to the vacuum chamber 1. When the UED is placed in abutting contact with the glans penis of a patient, the area defined by the interior of the vacuum chamber 1, the exterior of urethral extension tube 2 and the glans penis, is completely enclosed and may have the air within it evacuated to a specific sub-atmospheric pressure through the operation of the vacuum system. The vacuum applied acts to gently pull in the tissue of the glans penis that surrounds the urethra into the space between the outer adaptor ring 4 and the inner adaptor ring 5, thereby securing the UED and creating a leak-free seal between the UED and the glans penis. The interior of the urethral extension tube 2 remains at atmospheric pressure at all times during the use of the UED, thereby allowing for an unaltered measurement of the flow rate and pressure of the urine exiting the urethra during micturition.

The UED is preferably constructed of transparent material to facilitate accurate placement of the inner tube around the urethral orifice. All materials are also rigid so as not to introduce compliance to the system, and are inert to biological fluids such as urine or blood. The components of the UED are preferably constructed from poly(methyl methacrylate) (also known as Plexiglas®), but could also be constructed from polyethylene, polyurethane, polycarbonate, glass or another material exhibiting similar properties, such as polymeric materials used in medical devices such as lumens, tubes and catheters. The vacuum chamber 1 preferably has an outside diameter of approximately 2″ to 4″, and most preferably has an outside diameter of 2½ to 3″. The urethral extension tube 2 preferably has an outside diameter of approximately ½″ to 1½, and most preferably has an outside diameter of approximately 1″. The inner diameter of urethral extension tube 2 is preferably approximately ¼″. The inner diameter of the urethral extension tube 2 is sized so as to add no resistance to flow at the typical pressures encountered. Variations in dimensions allow for application on different size penises. It is also envisioned that other variations could involve inserting an appropriately shaped lumen into the closed end of the vacuum chamber to function in a similar fashion to the urethral extension tube 2. Another variation comprises a molded plastic disposable version of the UED that would be designed for a single use application.

In use, a water-based lubricant (such as KY jelly or similar material approved for tissue contact) is applied to the glans penis and to the surfaces of outer adaptor ring 4 and inner adaptor ring 5, and the UED is placed in abutting contact with the glans penis of the patient. The vacuum system 20 is activated and a sub-atmospheric vacuum is thereby applied to pull tissue into the space between outer adaptor ring 4 and inner adaptor ring 5. This acts to securely couple the glans penis to the UED and create a leak free seal with the urethral orifice. The connection is leak free as long as the urine pressure at the orifice does not exceed the vacuum applied. This requires that the vacuum be in excess of the maximum expected bladder pressure (120 mm Hg), yet less than the pressure likely to rupture capillaries in the glans and result in bruising. FDA approved vacuum constructor devices used for treatment of sexual impotence permit application of sub-atmospheric pressures of up to 300 mm Hg. These pressures may be sustained for up to 30 minutes without adverse consequences. The entire urodynamic testing requires less than 15 minutes from the time vacuum is applied to the penis.

The only consequence of use of the UED is a temporary ring of redness and swelling of the glans that usually disappears within a short time. Thus, this non-invasive apparatus is a vast improvement over the painful invasive tools currently used for diagnosis.

The female UED 11 is shown in FIG. 3. It is similar in concept and operation to the male unit, but modified to make a suction attachment to a concave tissue interface rather than convex as in the male. A female outer adaptor ring 12 and female inner adaptor ring 13 are shaped and positioned to conform to the tissue surrounding the urethral orifice of a female patient. The other components of the female UED are as shown for the male UED.

In addition, it is foreseen that variations of the UED could be utilized in other medical procedures, such as a colostomy, which require a leak-free seal and connection to a body opening.

The vacuum system 20, shown in FIG. 4, comprises a vacuum pump 21, a pressure regulator valve 22, a safety relief valve 23, a trap 24 and an input line 25, connected in series. The vacuum pump 21 is a standard, electrically powered vacuum pump that can generate sub-atmospheric pressures in excess of 400-500 mm Hg. The pressure regulator valve 22 can be set to limit the maximum vacuum in the system. In the embodiment shown in FIG. 4, the line from the regulator valve 22 goes to a trap 24 that removes any liquid in case of a leak, thereby protecting the pump and ensuring that there is no electrically conductive connection between the vacuum pump 21 and the UED. A safety relief valve 23 allows either manual or automatic release of the vacuum during a test in the unlikely event of subject discomfort. The input line 25 connects the vacuum system 20 to the UED.

In another embodiment, a holding tank of sufficient volume to maintain the vacuum during a test is placed between the pump and trap, allowing the pump to be turned off during a test and eliminating any line voltage operating during the test. In yet another embodiment, a hand-operated vacuum pump is used, similar to those used to test vacuum systems found in automobile engines. This can easily generate and hold the required vacuum and can be attached directly to the trap, eliminating need for an electric pump and regulator.

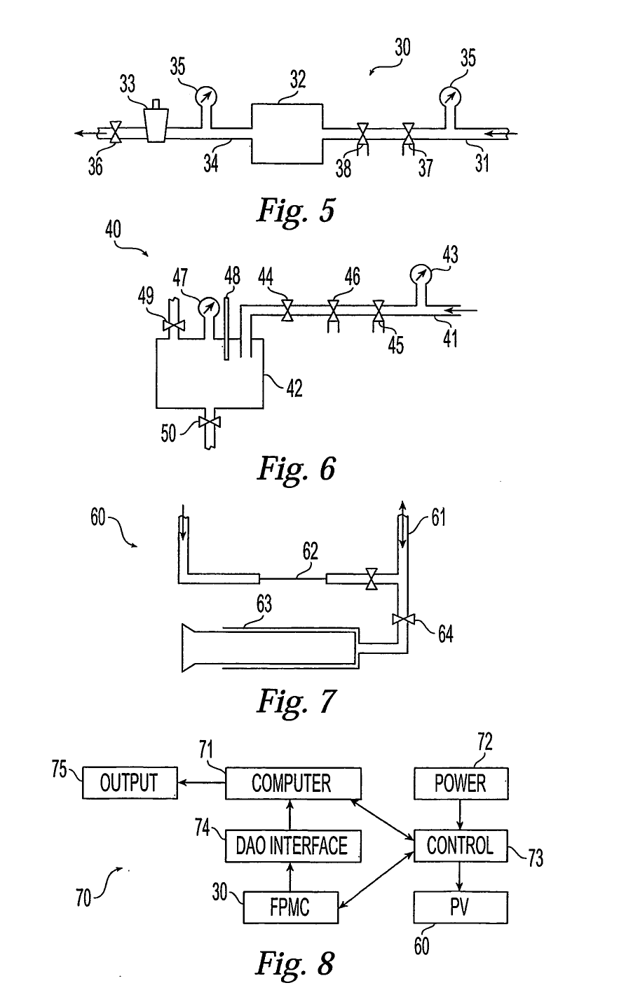

The flow and pressure measuring and control system 30 preferably comprises flow rate and pressure measurement instrumentation and control devices, interconnected by rigid tubing characterized by negligibly low pressure drop at maximum urine flow rates. As shown in FIG. 5, an input tube 31 is connected to and receives urine flow from the urethral extension tube 2 of the male UED 10 or the female UED 11. Input tube 31 is connected to the sensor of a sensitive, rapid response flow meter 32 to measure instantaneous flow rate. An electromagnetic flow meter, low pressure-drop orifice or similar meter, or any other flow measuring device that meets the requirements of rapid response and very small pressure drop can be used. A pressure control valve 33 is positioned on the output tube 34 that receives urine flow expelled from the flow meter 32. The pressure control valve 33 can be manipulated to maintain a set back pressure as required to modify the test conditions. The pressure control valve 33 can be operated by the control, acquisition and analysis system 70 (discussed below) in response to current conditions, or can be operated manually, as desired. Sensitive pressure transducers 35 (0-150 mm Hg) are positioned on the input tube 31 and the output tube 34 for monitoring the instantaneous pressure response of urine flowing through the system. A fast response on-off solenoid 36 or similar valve for very rapidly stopping or restarting flows during a test is positioned on output tube 34. The output tube 34 discharges urine into a suitable waste receptacle (not shown). The waste receptacle could be calibrated for volume so that the total volume of urine collected during a test can be measured independently of the flow measures. This would provide a useful clinical measure and also act as a check on the flow measures because the total integral of flow rate to time curve should equal the volume collected if the apparatus is properly calibrated.

A bypass output valve 37 and a bypass input valve 38 are located on input tube 31 to allow for the insertion of a parameter variation device 60 (discussed below) into the system if needed during a test. Valves 37 and 38 are 3-way solenoid or other electrically controllable valves, normally open to the line, but which may be utilized to redirect urine flow through the parameter variation device 60 before returning the urine flow back to the flow and pressure measuring and control system.

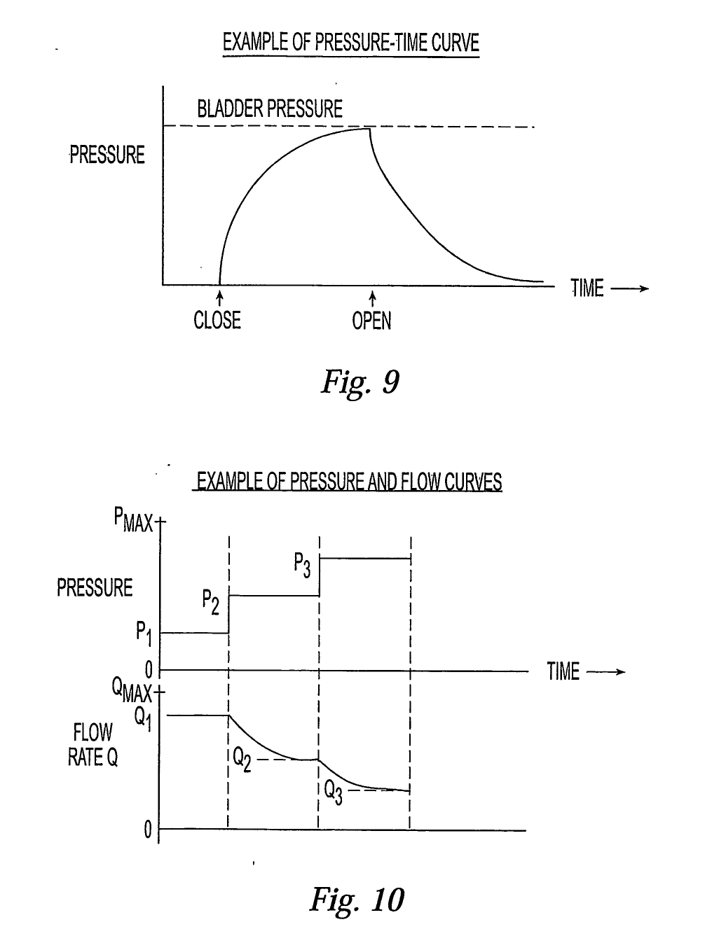

During a test, the various components may or may not be used, depending on the specific data to be collected. The system is first primed with saline to remove all gas, which, if present, can change the compliances to be measured. To determine bladder pressure, the pressure control valve 33 and fast response on-off solenoid 36 are opened fully. When urine flow is detected by the flow meter 32, fast response on-off solenoid 36 closes. The pressure measured by the sensitive pressure transducers 35 will rapidly rise until it reaches a steady value, which is the bladder pressure. If on-off solenoid 36 is now opened fully, the pressures will fall. FIG. 9 is a graph that illustrates the typical pressure-time relationship exhibited during a test. The pressure-time curves produced by these tests can be analyzed (see below) to obtain information about the resistances and compliances of the lower urinary tract. To measure specific parameters, such as the urethral resistance and compliance as a function of pressure or flow, pressure control valve 33 can be set to maintain specific pressures as the flow rate is measured, as illustrated in FIG. 10.

In an alternate embodiment, no flow meter is used. This embodiment needs no priming and uses thermodynamic and fluid mechanical properties of a gas-liquid system to determine the urine flow rate from gas phase pressure-time measurements. In this embodiment, as shown in FIG. 6, the flow and pressure measuring and control system 40 comprises an input tube 41 connected to a gas/liquid rigid chamber 42. The total volume of gas/liquid rigid chamber 42 is accurately known. The chamber 42 may initially contain no liquid, or it may contain a known volume of liquid to adjust the initial air volume. A sensitive pressure transducer 43 is positioned on the input tube 41 to measure the instantaneous pressure response of urine flowing through the system, and a solenoid valve 44 controls the flow of urine into the gas/liquid rigid chamber 42. A bypass output valve 45 and a bypass input valve 46 are located on input tube 41 to allow for the insertion of a parameter variation device 60 into the system, if needed during a test.

During a test, urine enters the system as described above for the preferred embodiment. After passing through input bypass valve 46, the flow passes through the normally open solenoid valve 44 and enters the gas/liquid rigid chamber 42, initially filled with air at atmospheric pressure. A transducer 47 and thermistor 48 are mounted in the gas/liquid rigid chamber 42 to measure gas pressure and temperature, respectively, in the chamber. Normally closed solenoid valve 49 may be opened to equalize the pressure within gas/liquid rigid chamber 42 with atmospheric pressure. Before a test is begun, valve 49 is briefly opened and closed, to ensure atmospheric pressure in chamber 42, as indicated by pressure transducer 47. Drain valve 50 is a solenoid valve that can be opened as needed to drain liquid to waste. As urine enters the gas/liquid rigid chamber 42, it pools at the bottom, and since the urine is incompressible, the air pressure in the chamber increases as a function of the volume of liquid collected. Under these conditions, air behaves as an ideal fluid thermodynamically and the mass of air trapped in the gas/liquid rigid chamber 42 can be calculated. Furthermore, at constant temperature and air mass, the pressure-volume product is constant. Therefore, as the chamber fills with liquid and the air contained within gas/liquid rigid chamber 42 is compressed, the air volume decrease is easily calculated from the increase in pressure. The liquid volume at time t=t(n) can therefore be calculated from difference between initial air volume and that calculated at time t=t(n). The flow rate can also be determined from the slope of the volume-time curve recorded from this analysis. Therefore, both the instantaneous pressure and urine flow rate are obtained from the same pressure-time curve, with no independent flow measurement being required.

Several test variants are possible and may be performed during the course of a single test. In one, the subject voids and valve 44 closes, thereby interrupting the urine flow and allowing a measurement of the static bladder pressure, as indicated by pressure transducer 43, to be taken. In another test, valve 44 is opened and urine flows into the gas/liquid rigid chamber 42, compressing the air as indicated by transducer 47. Again, this pressure rises to bladder pressure. At this point, valve 44 can be closed and drain valve 50 opened, draining all or some of the liquid and reducing the air pressure. Reopening valve 44 allows the test to be repeated at the same or different initial pressure. Typical output curves for these procedures are illustrated in FIG. 10. These time curves are a function of the subject's LUT resistances and compliances, the known resistances and compliances of the parameter variation device 60 (if used), and the time varying air compliance in gas/liquid rigid chamber 42. By using different initial air pressures, repeat runs can estimate system resistance and compliance as functions of different ranges of LUT pressures.

The parameter variation device 60, shown in FIG. 7, allows the insertion of a known resistance and/or compliance into the flow circuit comprising the bladder and urethra, when the urodynamic diagnostic device is attached to the subject. Since the compliance of a gas is proportional to its volume at a given pressure and temperature, the compliance of a system can be altered by adding a known volume of gas into the system. The parameter variation device 60 is available to change the dynamic response of the system in certain cases. Changing the overall flow system resistance and compliance changes the time rate of response of the pressure output during unsteady state test procedures and therefore permits more precise differentiation between urethral and bladder parameters. For example, if the subject has very low system compliance, inserting added compliances will increase the time constant of the response, allowing differentiation between bladder and urethral resistances or determination of a more precise value of the prostatic urethral resistance.

FIG. 7 shows the elements of the parameter variation device 60. Input tube 61 is connected to the bypass output valve 37 of the flow and pressure measuring and control system 30. Urine flow can be diverted via bypass output valve 37 to flow through resistance 62, thereby inserting a fluid resistance of known value into the system. The resistance 62 may comprise capillary tubes of known resistance, or may alternatively comprise a flexible tube that is compressed by a linear transformer controlled by the computer 71 (described below) to vary the flow resistance. The circuit compliance is varied by the variable compliance unit 63. Valve 64 can be opened to allow a variable volume of gas to be introduced into the system by introducing variable compliance unit 63 into the system. Variable compliance unit 63 preferably comprises a gas-tight syringe of appropriate volume that will allow reproducible volumes to be manually set prior to the test. However, any configuration that allows for the introduction of a known volume of gas into the system would be appropriate. For an automatic embodiment, a gas piston-cylinder device in which the piston is positioned by a linear stepper motor driven by the computer 71 could be utilized to allow changing system compliance during a test.

The control, data acquisition, and analysis system 70 serves to provide power to the components of the urodynamic diagnosis device and to control the operation of elements such as valves and transducers, in response to test criteria. It also captures the output of pressure and flow measuring elements, saves and analyzes the test results, and provides suitable output to the physician. As shown in schematic form in FIG. 8, computer 71 contains specific programs for control and analysis, and acts as an overall system manager. The preferred control program is the LABVIEW software program produced by National Instruments. However, other programs that are capable of interactively controlling the various components of the device would also be suitable. Power unit 72 provides power to the various transducers for measuring pressure and flow, as well as to the valves for controlling the system flow and pressure. Power unit 72 is preferably comprised of batteries with sufficient power to operate the equipment. Valves are preferably powered by a 12V automobile or motorcycle battery and transducers are powered by dry cell batteries. However, any embodiment that is a low-voltage, electrically isolated system (so that line voltage can not contact the subject in the unlikely event of direct electrical failure) is acceptable.

The control interface 73 contains the needed amplifiers and switches to operate the equipment during a test. The system operating program in the computer sends commands to the control interface, which then activates the necessary controls, such as valves. The data acquisition interface 74 samples and amplifies the signals as necessary for recording by the computer.

Diagnostic output 75 presents to the physician a urodynamic profile of the test subject. From the pressure and flow curves measured, the resistance and compliance that can be attributed to the bladder and urethra is calculated at various flows and pressures. Suitable mathematical models are then used for the dependence of resistance and compliance on flow rate and pressure to calculate parameters from the pressure and flow curves specific to the patient being tested. In this manner a fine-grained analysis of lower urinary tract function can be obtained from a series of non-invasive measures taken during a single episode of micturition.

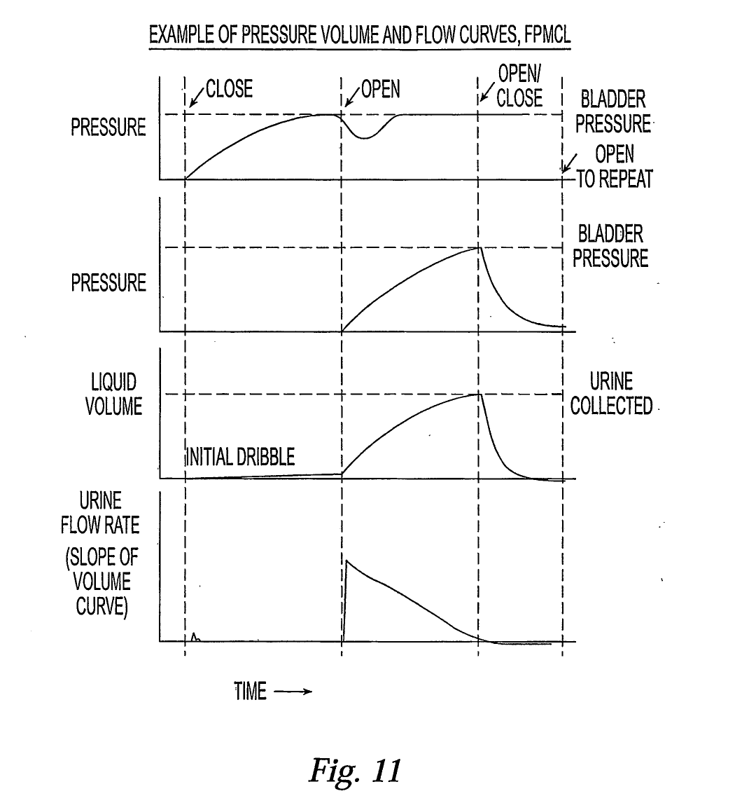

In a typical test, a specific sequence of operations is programmed into the computer 71 for opening and closing the necessary valves, and setting and calibrating the required transducers. Once the UED 10 is properly attached, the subject is asked to begin voiding. The system then operates automatically to carry out the test sequence that has been programmed: the flow meter 32 detects urine flow, signals the computer 71 which then signals the control interface 73 to operate the necessary valves in the proper test sequence. As the transducers measure the pressures and flow, the data acquisition interface 74 samples the signals and sends them to the computer 71 for storage. Examples of some typical test sequence that can be programmed and their output results are given in FIGS. 9, 10 and 11. The program may also have elements that branch depending on the data. For example, if an initial sequence shows a very rapid decay, the program may switch the parameter variation device 60 into the flow circuit to add resistance and/or capacitance, and then repeat the sequence. In this way multiple tests may be run during a single voiding episode.

The operation of this system and the parameters to be analyzed will be apparent to anyone familiar with the anatomy and physiology of the LUT, and with principles of fluid kinematics and dynamics of fluids in compliant tubes and vessels. When such a fluid filled system is static, i.e., no flow takes place, the pressure everywhere in the system is equal. The volume in this condition is a function of pressure since the compliant tubes stretch, thereby storing energy in their walls. When flow resumes, the walls relax, adding their energy to the fluid flow, so that the flow is driven both by the contracting bladder and relaxing compliances. The pressure falls from a maximum in the bladder to atmospheric pressure at the urethral orifice due to various flow resistances along the way and the properties of the compliant tissues. Flow resistance may increase in males due to BPH or in females due to bladder outlet obstruction. Tissue compliances may change due to a variety of conditions, and various pathological processes may affect bladder pressure. If flow is occluded, the pressure measured at the urethral orifice by suitable means will equal the bladder pressure. When the occlusion is removed, complex time dependent curves of pressure and flow rate result, which contain detailed information of all these parameters. Therefore, measurement of the pressure and flow rate at the urethral orifice as a function of time, and analysis of these functions using known principles of fluid dynamics with their associated mathematics, will yield parameters specific to the function of the LUT being tested at that time.

The entire testing process utilizing the urodynamic diagnosis device, once micturition begins, is controlled and recorded by the control, acquisition and analysis subsystem 70. In this manner, the device permits a rapid series of tests to be performed during a single episode of micturition. The examples given above are only a small fraction of those possible with this invention. Also, the specific items and layouts described herein and in the figures are for illustrative purposes only and are not meant to exhaust or exclude other configurations and components based on the same principles. Other possible tests can include static and dynamic measures of bladder pressure, as well as dynamic tests as described above to determine and evaluate bladder and urethral function, both independently and together. An additional test that is easily performed on males is to isolate the distal urethra by palpating the urethra at the base of the scrotum where it is distal to the prostate and lies just beneath the skin. By compressing and closing the urethra at that point, the only flows and pressures recorded will be those of the distal urethra, allowing urethral properties to be factored into prostatic and distal components, an advantage in differentiating types of urethral dysfunction.

When the test is complete, data is reduced using the analysis program within the computer, and/or saved on a CD for more detailed analysis at a later time. The analysis program is a major component of the overall invention, since it derives quantitative parameter values describing both bladder and urethral properties. By fitting a mathematical flow model to the data that takes into account their resistive and compliant properties, as well as by measuring bladder pressure, the analysis provides an objective basis to carry out a differential diagnosis.

In the simplest model, the entire LUT is represented by one resistance and one compliance. For simplicity, assume that these values are constant and independent of pressure and flow. This results in a “first-order dynamic system,” whose mathematical solution is well known: the pressure P will increase with time t by an exponential function governed by a single parameter called the time constant ô, which is the product of the resistance R and compliance C, i.e.,

$$P = {P}_{o} [ 1 - exp ( - t / ô ) ] \qquad ô = RC , \qquad {P}_{o} = pressure at zero flow$$

If the model were valid, this equation could describe the curve in FIG. 9 obtained when on-off valve 36 is closed, from which would be derived the bladder pressure and the time constant. A similar equation with the same time constant would describe the curve when the valve is reopened; either would yield the value of the RC product. To obtain individual values of R and C, one can either conduct a steady flow test, in which both pressure and flow are measured, giving R, or insert a known resistance into the flow line, using the PV subsystem, giving a different value of the time constant; the two ô values will yield values for R and C.

However, a first order system with constant parameters is not valid. The resistance is a function of pressure because the dimensions of a compliant system vary with pressure. Also, the LUT will have different time constants for the bladder and the urethra, each with distinct R and C components that are complex, non-linear functions of flow rate and pressure, resulting in a second-order dynamic system. Such a system requires two exponential terms even if the parameters are constant and requires solving a differential equation with coefficients that are functions of pressure and/or flow rate. Such an equation can be solved on a computer using various known numerical techniques. By considering the types of functions that best describe the pressure and flow in the LUT, the solution can be optimized so that individual subject parameters can be obtained from the results of the different tests possible with the apparatus described. These parameters can be selected based on knowledge of the physiology and pathophysiology of the LUT, and from experience by testing with normal subjects and subjects with known LUT dysfunction. For example, consider the type of test shown in FIG. 10. A sequence of steady pressures and flows, each separated by a pressure jump with exponential-type flow relation will yield a data series from which values of resistance and compliance can be calculated as a function of pressure. With experience, the ability to analyze the same data using progressively more sophisticated models will allow the most efficient model to be selected for specific diagnostic applications. Furthermore, since the model parameters describe a specific subject on a given date, the ability to repeat these tests during and after a course of treatment presents objective data with which to follow and evaluate the results of the treatment.

The overall utility and applicability of the invention relates to the general concepts presented, and is not dependent on or restricted to the specific embodiments presented above primarily for purposes of explication and illustration. The overall scope of the invention is given by the appended claims, and any other embodiments or changes to the apparatus or procedures that fall within the meaning of the claims are considered to be within their scope.

What is claimed is:

1. A urethral extender device for providing a non-invasive, leak free attachment to a human male urethra, comprising:
a vacuum chamber having a proximal end, a distal end, a vacuum outlet port and an outer adaptor ring positioned at said proximal end;
a urethral extension tube with an inner adaptor ring, coupled to said vacuum chamber distal end and extending through said vacuum chamber; and
wherein said outer adaptor ring and said inner adaptor ring are shaped and positioned to hold a glans penis immobile and to provide a leak free seal to the urethra, while allowing for unimpeded urine flow through said urethral extension tube.

2. The urethral extender device of claim 1, wherein:
said outer adaptor ring and said inner adaptor ring come into abutting contact against said glans penis; and
a vacuum is applied to said vacuum outlet port thereby sealing said outer and inner adaptor rings securely against said glans penis.

3. The urethral extender device of claim 2, wherein said urethral extender device comes into contact only with said glans penis.

4. The urethral extender device of claim 1, wherein said urethral extension tube is slidably coupled to said vacuum chamber.

5. The urethral extender device of claim 4, additionally comprising means for locking said slidably coupled urethral extension tube in place.

6. The urethral extender device of claim 1 wherein said vacuum chamber is formed from a transparent, non-compliant material.

7. The urethral extender device of claim 1, wherein said vacuum chamber is formed from material selected from the group consisting of poly(methyl methacrylate), polyethylene, polyurethane, polycarbonate, and glass.

8. The urethral extender device of claim 1, wherein said vacuum chamber and said urethral extension tube are cylindrical in shape.

9. The urethral extender device of claim 8, wherein said vacuum chamber has an outside diameter of between 2½ in. to 3 in.

10. The urethral extender device of claim 8, wherein said urethral extension tube has an outside diameter of between ½ in. to 1½ in.

11. The urethral extender device of claim 2, wherein a vacuum of up to 300 mm Hg. is applied to said vacuum outlet port.

12. A urethral extender device for providing a non-invasive, leak free attachment to a human female urethra, comprising:
a vacuum chamber having a proximal end, a distal end, a vacuum outlet port, and an outer adaptor ring positioned at said proximal end;
a urethral extension tube including an inner adaptor ring, coupled to said vacuum chamber distal end and extending through said vacuum chamber;
wherein said outer adaptor ring and said inner adaptor ring are shaped and positioned to conform to tissue adjacent to a urethral orifice; and
wherein said outer adaptor ring and said inner adaptor ring provide a leak free seal to the urethra, while allowing for unimpeded urine flow through said urethral extension tube.

13. The urethral extender device of claim 12, wherein:
said outer adaptor ring and said inner adaptor ring come into abutting contact against said tissue adjacent to a urethral orifice; and
a vacuum is applied to said vacuum outlet port, thereby sealing said outer and inner adaptor rings securely against said tissue surrounding a urethral orifice.

14. A method for utilizing a urethral extender device to allow for unobstructed urine flow from a male patient into an analysis device, comprising:
connecting a vacuum source to a urethral extender device that fits in abutting contact with the surface of a glans penis of a patient and allows for unobstructed urine flow through said urethral extender device into an analysis device;
placing said urethral extender device against the glans penis of a patient; and
activating said vacuum source to produce a vacuum inside said urethral extender device, thereby sealing said urethral extender device against said glans penis, to provide a leak-free seal with said glans penis while simultaneously allowing for unobstructed urine flow through said urethral extender device into an analysis device.

15. A method for utilizing a urethral extender device to allow for unobstructed urine flow from a female patient into an analysis device, comprising:
connecting a vacuum source to a urethral extender device that fits in abutting contact with tissue adjacent to a urethral orifice of a patient and allows for unimpeded urine flow through said urethral extender device;
placing said urethral extender device against tissue surrounding said urethral orifice of a patient;
activating said vacuum source to produce a vacuum inside said urethral extender device, thereby sealing said urethral extender device to said tissue surrounding the urethra of a patient, to provide a leak-free seal with said tissue while simultaneously allowing for unobstructed urine flow through said urethral extender device into an analysis device.

16. A flow and pressure measurement apparatus for measuring the unobstructed flow rate and pressure of urine flow, comprising:
an input tube and an output tube, each having a proximal end and a distal end;
a first pressure transducer fixed to said input tube, a flow meter connected to said input tube distal end, and said output tube proximal end additionally connected to said flow meter;
a bypass input valve and a bypass output valve connected to said input tube between said first pressure transducer and said flow meter;
a pressure control valve and a second pressure transducer fixed to said output tube; and
a fast response valve fixed to said output tube distal end.

17. The flow and pressure measurement apparatus of claim 16, wherein said apparatus is utilized for performing a lower urinary tract diagnostic examination.

18. The flow and pressure measurement apparatus of claim 17, additionally comprising a urethral extender device connected to said input tube, that fits in abutting contact against the surface of a glans penis of a patient and allows for unobstructed urine flow through said urethral extender device into said flow and pressure measurement apparatus.

19. The flow and pressure measurement apparatus of claim 18, wherein said flow meter is an electromagnetic flow meter.

20. The flow and pressure measurement apparatus of claim 18, wherein said flow meter is a low pressure drop liquid flow meter.

21. The flow and pressure measurement apparatus of claim 18, wherein said flow meter is a low pressure-drop capillary flow meter.

22. The flow and pressure measurement apparatus of claim 18, wherein said valves are electrically controllable.

23. The flow and pressure measurement apparatus of claim 22, wherein said bypass input valve and said bypass output valves are three-way solenoid valves.

24. The flow and pressure measurement apparatus of claim 22, wherein said fast response valve is an on-off solenoid valve.

25. A flow and pressure measurement apparatus for measuring the unobstructed flow rate and pressure of urine flow, comprising:
an input tube having a proximal end and a distal end;
a first pressure transducer fixed to said input tube proximal end;
a fast response valve connected to said input tube;
a bypass input valve and a bypass output valve connected to said input tube between said first pressure transducer and said fast response valve; and
a rigid chamber with a pressure relief valve, a transducer and a thermistor, connected to the distal end of said input tube.

26. The flow and pressure measurement apparatus of claim 25, wherein said apparatus is utilized for performing a lower urinary tract diagnostic examination.

27. The flow and pressure measurement apparatus of claim 26, additionally comprising a urethral extender device, connected to said input tube, that fits in abutting contact against the surface of a glans penis of a patient and allows for unobstructed urine flow through said urethral extender device into said flow and pressure measurement apparatus.

28. The flow and pressure measurement apparatus of claim 27, wherein said valves are electrically controllable.

29. The flow and pressure measurement apparatus of claim 28, wherein said bypass input valve and said bypass output valves are three-way solenoid valves.

30. The flow and pressure measurement apparatus of claim 28, wherein said fast response valve is an on-off solenoid valve.

31. A control, data acquisition and analysis apparatus comprising:
a computer with program software and a data acquisition interface;
a control interface in communication with said computer; and
a power unit connected to and controlled by said control interface;
wherein:
said computer monitors and controls a flow and pressure measurement device in communication with said control, data acquisition and analysis apparatus, to perform a lower urinary tract diagnostic examination.

32. The control, data acquisition and analysis apparatus of claim 31, additionally comprising a urethral extender device connected to said flow and pressure measurement device, that fits in abutting contact against the surface of a glans penis of a patient, and allows for unobstructed urine flow through said urethral extender device into said flow and pressure measurement device.

33. The control, data acquisition and analysis apparatus of claim 32, wherein said computer records data and provides an analysis of the data received from said flow and pressure measurement system.

34. The control, data acquisition and analysis apparatus of claim 33, wherein said power unit comprises a 12 volt storage battery.

35. A parameter variation apparatus for a flow and pressure measurement device comprising:
an input tube with a proximal end and a distal end;
a resistance with an input and an output, wherein said input is connected to said distal end of said input tube;
a variable compliance unit connected between said input tube proximal end and said resistance;
an output tube connected to said output of said resistance;
wherein:
said input tube proximal end and said output tube are in connection with a flow and pressure measurement device to allow said parameter variation apparatus to vary the flow rate and pressure of a urine flow through said flow and pressure measurement device, thereby facilitating a diagnostic examination of a lower urinary tract.

36. The parameter variation apparatus of claim 35, wherein said resistance comprises a capillary tube of known resistance.

37. The parameter variation apparatus of claim 35, additionally comprising a linear transformer;
wherein:
said resistance comprises a flexible tube; and
said linear transformer compresses said flexible tube to vary the flow resistance.

38. The parameter variation apparatus of claim 35, wherein said variable compliance unit comprises a chamber with a variable volume.

39. The parameter variation apparatus of claim 35, wherein said variable compliance unit comprises a piston-cylinder device driven by a linear stepper motor to adjust the volume of said variable compliance unit.

40. A urodynamic diagnosis device for conducting a non-invasive examination of a lower urinary tract by analyzing the flow and pressure of urine released during micturition, comprising:
a urethral extender device, with a urethral extension tube and a vacuum outlet port, that fits in abutting contact against the surface of a glans penis of a patient and allows for unobstructed urine flow through said urethral extension tube;
a vacuum system connected to said urethral extender device vacuum outlet port;
a flow and pressure measurement device connected to said urethral extension tube; and
a control, data acquisition and analysis system in communication with said flow and pressure measurement system to perform a diagnostic examination of a lower urinary tract.

41. The urodynamic diagnosis device of claim 40, additionally comprising a parameter variation circuit connected to said flow and pressure measurement system to alter the flow and pressure of urine through said flow and pressure measurement system.

42. The urodynamic diagnosis device of claim 40, wherein said vacuum system comprises an electric vacuum pump.

43. The urodynamic diagnosis device of claim 42, wherein said vacuum system additionally comprises a vacuum holding tank to maintain a vacuum during a test.

44. The urodynamic diagnosis device of claim 40, wherein said vacuum system comprises a hand-operated vacuum pump.

45. The urodynamic diagnosis device of claim 40, wherein said control, data acquisition and analysis system additionally comprises a computer with software means for performing and analyzing the results of a lower urinary tract diagnostic examination.

46. A urodynamic diagnosis device for conducting a non-invasive examination of a lower urinary tract, comprising:
a urethral extension tube;
means for non-invasively forming a leak free seal between said urethral extension tube and tissue surrounding a urethra of a patient;
means for measuring the instantaneous pressure and flow rate of urine released by said patient; and
means for analyzing the relationship of said pressure to said flow rate, to determine the source of lower urinary tract dysfunction.

47. A method for non-invasively diagnosing dysfunction of the bladder, urethra or prostate, comprising:
attaching to a urethra a vacuum sealed urine flow rate and pressure measurement device having means for measuring urine flow rate and pressure; and
measuring urine flow rate and pressure upon micturition.

48. A method for treating bladder disease or prostate disease, comprising:
measuring an unaltered urine flow rate and pressure using a vacuum sealed urine flow rate and pressure measuring device, non-invasively connected to a urethra of a patient; and
choosing an appropriate therapy for said patient based upon an analysis of the measured flow rate and pressure.

49. A method for treating decreased urine flow in a patient, comprising:
securing a non-invasive measuring device to a urethra of a patient and allowing said patient to begin urination;
measuring the unaltered pressure and flow rate of urine exiting from said urethra;
recording said pressure and flow rate measurements as a function of time;
analyzing said pressure and flow rate measurements to determine a cause for a decrease in urine flow of a patient; and
prescribing an appropriate treatment to restore normal urine flow.

50. A method for making an early diagnosis of bladder outlet obstruction utilizing a urodynamic diagnosis device, comprising:
securing a urine measuring device to tissue surrounding a urethra of a patient without entering said urethra;
urinating into said urine measuring device;
measuring an unaltered pressure and flow rate of urine exiting said urethra;
recording said pressure and flow rate; and
analyzing said pressure and flow rate measurements to detect the presence of a bladder outlet obstruction.

51. A method for differentiating dysfunction of the bladder from dysfunction of the prostate, where either of which might create the symptom of urination irregularity, comprising:
measuring an unaltered pressure and flow rate of urine exiting a urethra of a patient;
recording said pressure and flow rate; and
analyzing said pressure and flow rate to differentiate bladder dysfunction from prostate dysfunction.
